(19) United States
(12) Patent Application Publication
(10) Pub. No.: US 2021/0128327 A1
(43) Pub. Date: May 6, 2021

(54) MEDICAL DEVICE FOR PRODUCING A DATA MODEL OF A LIMB STUMP, SET, METHOD AND RETAINING STOCKING OR SENSOR ASSEMBLY

(71) Applicant: Romedis GmbH, Neubeuern (DE)

(72) Inventor: Andreas RADSPIELER, Neubeuern (DE)

(73) Assignee: Romedis GmbH, Neubeuen (DE)

(21) Appl. No.: 16/618,646

(22) PCT Filed: Jun. 21, 2018
(86) PCT No.: PCT/EP2018/066636

§ 371 Date: Dec. 2, 2019

(30) Foreign Application Priority Data
Jun. 23, 2017 (DE) 10 2017 114 001.8
Jan. 14, 2018 (DE) 10 2018 100 704.3

(51) Int. Cl. A61F 2/80; A61F 2/50

(52) U.S. Cl. CPC A61F 2/80; A61F 2/5046; A61F 2002/5049; A61F 2002/505; A61F 2002/5052

(57) ABSTRACT

A medical apparatus for use in generating a data model of a limb stump is provided, wherein the apparatus includes at least a pressure vessel with a fluid chamber or a pressure chamber for receiving or storing a fluid, wherein the pressure vessel includes a wall made of a first material, wherein the wall limits an interior of the pressure vessel against an exterior, and wherein the pressure vessel includes an insertion opening for inserting the limb stump into the interior of the pressure vessel. The apparatus further includes a fluid-impermeable membrane made of a second material and arranged to form or limit the fluid chamber or the pressure chamber. Furthermore, the apparatus comprises an adherent stocking or another sensor arrangement, to be pulled over the limb stump, wherein the adherent stocking includes sensors for generating the data model.

The present invention relates to an apparatus according to the preamble of claim 1. It further relates to a set according to the preamble of claim 17, a method according to the preambles of claim 19, as well as an adherent stocking or a sensor arrangement according to the preamble of claim 22.

Leg amputees may regain mobility using leg prostheses. Modern leg prostheses include various modules (prosthesis shaft, knee, lower leg and foot modules), which may be combined to meet the various needs of the prosthesis wearer (hereinafter referred to in short as wearer or patient) in terms of fundamental mobility, sport activities and aesthetic perceptions.

The module prosthesis shaft represents the connection between the mechanical replacement of the extremity and the residual limb stump (in short also referred to as stump) of the prosthesis wearer, e.g. a lower leg stump, a thigh stump or an arm stump. In the state of the art, the prosthesis shaft is individually adapted to the stump of the eventual wearer. For this purpose, often, a plaster impression is produced by a moist plaster bandage, which will be the basis of the prosthesis shaft and determines its shape substantially. Alternatively, the form of the limb stump may be digitally captured to subsequently produce the prosthesis shaft based on these digital data.

It is an object of the present invention to propose an apparatus for use in generating a data model of a limb stump, in particular of the lower leg stump. A further object of the present invention is to propose a set and a method for generating a data model of a limb stump. A further object of the present invention is to propose an adherent stocking or a sensor arrangement for a limb stump.

The object according to the present invention may be achieved by an apparatus (or a system) having the features of claim 1. It may further be achieved by a set having the features of claim 12, by a method having the features of claim 19, in addition by an adherent stocking or a sensor arrangement having the features of claim 22.

A medical apparatus (in short: apparatus) is thus provided by the present invention which may be used in the manufacturing of a prosthesis shaft for a limb stump in particular a lower leg stump. This may, for example, be used for manufacturing a lower leg prosthesis shaft, on the, only preferably standing, patient.

In this, the apparatus comprises a fluid container or a pressure vessel with exactly or at least one fluid chamber. This may receive or store an optionally pressurized fluid. Thereby, the pressure is above the atmospheric pressure. The fluid is a gas or a liquid, preferably air or water, since the latter two are respectively cheap and easily available.

The pressure vessel comprises a wall, which is made of at least or exactly one first material, or comprises at least one first material.

The wall of the pressure vessel limits its interior. According to the present invention, the interior of the pressure vessel is understood to be the space or volume defined by the geometry of the pressure vessel or encompassed or circumscribed by an outer wall of the pressure vessel. If the pressure vessel is for example cylindrical, the interior of the pressure vessel is the space delimited by the cylindrical shell surface and by the two end sides or end planes. If the pressure vessel is, in another example, rectangular, the space of the interior is defined by the result of the multiplication of the height, width and depth of the rectangle. In determining the interior, it is irrelevant whether or not the space corresponding to the interior is fluid-tight. The interior does not represent or is not a fluid-tight closed space but rather a volume circumscribed by the wall. The space which does not belong to the interior of the pressure vessel is referred to herein as its exterior.

The pressure vessel comprises an insertion opening through which the limb stump (which may in this respect, herein also to be understood as the distal end of the stump instead of the whole stump) may be inserted into the interior of the pressure vessel. The insertion opening may, for example, be an open-end side or end plane, a passage through-opening in the wall or an opening which breaks through or interrupts the wall. In the region of the insertion opening, the interior is thus not separated from the exterior of the pressure vessel by a section of the wall. The insertion opening may lie in an insertion opening or in an insertion plane, through which the limb stump is inserted into the interior of the pressure vessel.

Furthermore, the pressure vessel comprises at least one or exactly one fluid-tight membrane. Alternatively, the pressure vessel does not have such a membrane as described further below, but only correspondingly suitable and/or provided receiving devices (such as the connector described in the following as optional) for receiving the membrane on/at the pressure vessel.

The membrane is made of or comprises a second material. The first and the second material differ from each other.

The wall is designed as a single-piece or optionally as a multi-piece. A multi-piece wall may comprise a plurality of movable/displaceable sections, in order to achieve a small packaging volume of the device for transport purposes, by way of example. The medical apparatus according to the present invention with the multi-piece wall may be referred to as a mobile medical apparatus.

Furthermore, the apparatus (or the system) according to the present invention comprises optionally at least one adherent stocking or one arrangement of sensors or sensor arrangement, each to be pulled and/or put on the limb stump. The adherent stocking thus connected to the limb stump or the sensor arrangement abutting the limb stump comprises optional sensors for generating a data model. In some embodiments, the apparatus according to the present invention comprises no adherent stocking or it comprises an adherent stocking which however comprises no sensors.

The set according to the present invention comprises at least one medical apparatus according to the present invention. It further comprises at least one transmitting device and/or one receiving device for transmitting and/or receiving signals of the sensors which are arranged in the adherent stocking or in the sensor arrangement.

The method according to the present invention serves for generating a data model of a limb stump. In this, a medical apparatus according to the present invention is provided. The method encompasses the step of capturing signals of the adherent stocking or of the sensor arrangement.

The adherent stocking according to the present invention or the sensor arrangement is provided and/or prepared to be pulled over a limb stump (KS) when generating a data model of the limb stump (KS).

In addition, each optionally, the pressure chamber is filled with a liquid, a balloon-like closed membrane will be or is provided inside the pressure vessel or the liquid level within the pressure chamber will be or is adjusted or changed in such a way that the membrane is covered by liquid at least in sections thereof around the entire circumference of these sections or is bulging beyond the insertion opening into the exterior of the pressure vessel.

Embodiments according to the present invention of each of the aforementioned subject-matters may comprise one or several of the following features in any combination unless the person skilled in the art recognizes a concrete combination as technically impossible. Also the subject-matters of the dependent claims each indicate embodiments according to the present invention.

In all of the aforementioned and following statements, the use of the expression “may be” and “may have” etc. is to understood synonymously with “is preferably” or “has preferably,” and so on respectively, and is intended to illustrate embodiments according to the present invention.

Whenever numerical words are mentioned herein, the person skilled in the art will recognize or understand them as indications of numerical lower limits. Unless it leads the person skilled in the art to an evident contradiction, the person skilled in the art will comprehend the specification for example of “one” encompassing “at least one”. This understanding is also equally encompassed by the present invention as the interpretation that a numerical word, for example, “one” may alternatively mean “exactly one”, wherever this is evidently technically possible for the person skilled in the art. Both are encompassed by the present invention and apply to all numerical words used herein.

In case of doubt, the person skilled in the art will understand spatial information like “top”, “bottom”, “upper” or “lower”, whenever they are herein mentioned, as a spatial indication with reference to the alignment in the figures appended hereto and/or of the arrangement of the apparatus(es) according to the present invention when used as intended.

What is herein stated about the adherent stocking applies as well for the herein disclosed sensor arrangement. This applies in particular as long as the interchangeability of the terms “adherent stocking” and “sensor arrangement” is not recognized by the person skilled in the art as being technically impossible.

An adherent stocking may in some exemplary embodiments of the present invention be a composite of material straps, material rings, and the like. The adherent stocking, when being used, needs not be designed to cover the limb stump completely or gapless around both the circumference of the adherent stocking and along its length.

The pressure vessel and/or the fluid-impermeable or fluid-tight membrane may correspond to the pressure vessel and/or to the fluid-impermeable membrane, as filed on 25 Jan. 2016 with the GPTO (German Patent and Trademark Office) by the present applicant under DE 10 2016 101 257.2 entitled “Apparatus, adapter, system and method for creating a plaster impression of a patient's limb stump for manufacturing a prosthesis shaft, in particular for the lower extremity”. The relevant disclosure of this application is hereby incorporated by reference in its entirety, provided that details or a concrete combination is not recognized by the person skilled in the art as being obviously technically impossible.

In specific exemplary embodiments according to the present invention, the pressure chamber is a locked and/or lockable space in which the fluid may be subjected to pressure above the atmospheric pressure (in short: atmosphere) without being able to escape from this space.

In some exemplary embodiments according to the present invention, the membrane is arranged to form the pressure chamber or alternatively to delimit it, e.g., by being part, in particular by being elastic or only in one direction elastic part, of the wall of the pressure chamber.

In specific exemplary embodiments according to the present invention, the membrane serves for building, by itself (e.g. in form of a balloon) or alternatively together with sections of the wall, a fluid-tight fluid chamber of the pressure vessel which chamber lies at least partially or completely in the interior of the pressure vessel. Since it can receive and/or hold fluid under a pressure which is above atmospheric pressure, this fluid chamber is designated herein as pressure chamber.

The terms “fluid chamber” and “pressure chamber” are interchangeable in certain exemplary embodiments according to the present invention and/or in those embodiments in which the person skilled in the art does not object thereto. What is said herein about the “pressure chamber” may also be applicable to a “fluid chamber”.

In some exemplary embodiments according to the present invention, the pressure chamber then lies inside the pressure vessel when the pressure prevailing in the pressure chamber does not exceed a certain pressure. Deviating from this, in some embodiments according to the present invention, the pressure chamber extends also to the exterior of the pressure vessel when the pressure prevailing in the pressure chamber pressurizes the membrane such that the latter bulges outwards, for example through the insertion opening, i.e. into the exterior of the pressure vessel. The pressure chamber may thus have a variable volume, which depends on the pressure prevailing in the pressure chamber. The latter does not apply to the constant interior the pressure vessel.

In some exemplary embodiments according to the present invention, a fluid may be maintained under a pressure above the atmosphere in the pressure chamber, regardless of the insertion opening penetrating through the wall.

In specific exemplary embodiments according to the present invention, the pressure chamber serves for receiving or supporting the distal end of the patient's limb stump which is inserted in the interior of the pressure vessel. Due to the fluid contained in the pressure chamber, the membrane nestles laterally or circumferentially to the distal end of the limb stump, which is covered by the adherent stocking or by the sensor arrangement, or to the entire limb stump. In this way, it may be possible to pressurize the limb stump through the membrane with the —referring to an area unit—preferably unchanged or same pressure.

In some exemplary embodiments according to the present invention, the pressure chamber is a fluid-tight closed space which is entirely, or among others, formed or limited by the wall of the pressure vessel and the membrane. In some exemplary embodiments according to the present invention, the term “pressure chamber” may be replaced by the definition above.

In some exemplary embodiments according to the present invention, the pressure vessel is the vessel or space in which the pressure chamber is arranged.

In some exemplary embodiments according to the present invention, the pressure vessel is a water vessel.

In so some exemplary embodiments according to the present invention, the term “pressure vessel” may be replaced by the term “fluid vessel” or “water vessel”.

In specific exemplary embodiments according to the present invention, the pressure vessel has a cylindrical shape.

In some exemplary embodiments according to the present invention, the pressure chamber is designed and/or arranged in such a way that the pressure prevailing therein depends, inter alia or exclusively, on the insertion depth of the limb stump into the interior of the pressure vessel, in any case during the intended use of the apparatus and with closed inlets and outlets, if present.

In certain exemplary embodiments according to the present invention, the membrane is arranged in the region of the insertion opening and is optionally there connected directly or indirectly to the wall in a releasable or non-releasable manner, preferably fluid-tight. It closes the insertion opening preferably similar to a cap, insofar the pressure present in the pressure chamber does not deviate significantly from the atmospheric pressure.

In some exemplary embodiments according to the present invention, the membrane is arranged to prevent a fluid or material exchange in the interior of the pressure vessel in its axial direction.

In certain exemplary embodiments according to the present invention, the membrane is always arranged in single-layer in axial and/or radial direction.

In some exemplary embodiments according to the present invention, the membrane is directly or indirectly connected to the pressure vessel on a first end side of the pressure vessel, but not also to a second end side opposite to the first end side.

In certain exemplary embodiments according to the present invention, the membrane is flat or balloon-like (i.e. open at one end), but not tube-like (i.e. open at both ends).

In some exemplary embodiments according to the present invention, the membrane is, at least partially, designed as a sealing element, preferably at its edge, e.g. as a sealing ring. In these embodiments, for example in the area of the insertion opening, the membrane is arranged around the limb stump. It optionally prevents the fluid, which is present in the pressure chamber, from losing pressure along the limb stump. It may advantageously prevent an outflow of fluid into the exterior of the pressure chamber and thus prevent a pressure drop inside the pressure chamber.

In some exemplary embodiments according to the present invention, the membrane is arranged inside the pressure vessel, at least during use of the apparatus (i.e. when the limb stump of the patient is inserted in the interior of the pressure vessel) and at least in sections thereof. Preferably, it is present only and/or always in the interior of the pressure vessel. Alternatively or additionally, it is connected directly or indirectly in fluid-tight connection to sections of the wall of the pressure vessel.

In some exemplary embodiments according to the present invention, the membrane is also present exclusively inside the pressure vessel during use of the apparatus (i.e. when the limb stump of the patient is inserted in the interior of the pressure vessel).

In some exemplary embodiments according to the present invention, the membrane does not protrude from the interior of the pressure vessel, in particular not in the area of a second end side or in the area of the bottom plane.

In some exemplary embodiments according to the present invention, the membrane is permanently connected to the apparatus. In certain embodiments according to the present invention, permanently means that the membrane cannot be detached from the pressure vessel without the use of tools or only destructively; for example, it can be permanently and yet releasably connected to the wall by a clamping ring or by clamping rings and one or more screws. A releasability using a tool may advantageously be provided in order to allow replacing the membrane, e.g., due to abrasion after a plurality of uses. In these embodiments, however, the membrane is not intended to be releasable from the pressure vessel by simple pulling over, pulling down or the like. At the same time, the permanent fastening may advantageously ensure that the forces which are transmitted in the pressure vessel by the fluid to the membrane during use of the apparatus cannot release the membrane from the pressure vessel or from its wall.

In some exemplary embodiments according to the present invention, at least one surface of the membrane and/or of the adherent stocking or the sensor arrangement is, at least in sections, coated with or consists of or carries a friction-reducing material (for example applied by lubrication, spraying, or the like of the membrane). This allows the patient to insert the limb stump sufficiently deep through the insertion opening into the interior of the pressure vessel. In this way, it is advantageously ensured that the membrane is not or is not excessively laterally displaced along the limb stump in a proximal direction. In turn this advantageously ensures that the membrane does not protrude proximally.

In some exemplary embodiments according to the present invention, the apparatus comprises no other axial receptacle for the free stump end than the membrane and/or no “axial reference compliant means”. The stump end contacts preferably only the membrane. In other embodiments according to the present invention this may be different.

In some exemplary embodiments according to the present invention, the apparatus according to the present invention comprises no, in particular circular, disk-shaped cover of the insertion opening, which is, e.g., made of rubber and/or which is optionally a single-piece with an integral central hole.

In some exemplary embodiments according to the present invention, the apparatus according to the present invention, comprises, in particular during its use, no sand, no plaster material, no curing material, in particular not in the pressure chamber or between wall and membrane.

In certain exemplary embodiments according to the present invention, the fluid is not sand, nor solid particles nor balls, in particular not polystyrene balls, or does not comprise suchlike.

In certain exemplary embodiments according to the present invention, the membrane is not made of, or comprises no, polyethylene.

In some exemplary embodiments according to the present invention, the apparatus according to the present invention, comprises no device, such as an elastic ring or a rubber ring, and in particular no rubber ring which is provided for fixing the membrane to an outer wall of the pressure vessel.

In other embodiments according to the present invention this may be different.

In some exemplary embodiments according to the present invention, the apparatus according to the present invention comprises no vacuum source (in particular no vacuum source which is operated electrically or hydraulically) or is not connected to such in fluid communication.

In certain exemplary embodiments according to the present invention, the apparatus according to the present invention comprises no pressure source and/or no, in particular inflatable, expansion devices or other “expander means” or is not connected to such.

In some exemplary embodiments according to the present invention, the apparatus according to the present invention comprises no vacuum connection.

In certain exemplary embodiments according to the present invention, the apparatus according to the present invention comprises no air chamber.

In some exemplary embodiments according to the present invention, the apparatus according to the present invention comprises no feed screws.

In some exemplary embodiments according to the present invention, the inlets and/or outlets which are provided with a closure device are part of the wall.

In certain exemplary embodiments according to the present invention, the pressure chamber is designed exclusively by the membrane and parts or sections of the wall, or exclusively by the membrane and parts or sections of the wall and fluid-tight connections between membrane and wall.

In some exemplary embodiments according to the present invention, an end side of the pressure vessel belongs to the wall, in particular the end side denoted herein as second end side.

In some exemplary embodiments according to the present invention, the pressure vessel is closed or closable in a fluid-tight manner at its second end side.

The pressure vessel may comprise a first and a second end side. The insertion opening may be an opening on an end side. Preferably, it is situated in the first end side or in the area of the first end side.

The membrane may be connected releasably or non-releasably to the wall or to another section of the pressure vessel.

The pressure chamber may be a space of the pressure vessel which is closed by the wall and the membrane. The pressure chamber is partially or completely inside the pressure vessel. A fluid stored in the pressure chamber may be trapped by the combination alone between the wall (as long as possibly provided openings are closed by the provided closures, valves, stopcocks, etc., each according to the present invention) and the membrane.

In some exemplary embodiments according to the present invention, the multi-part wall of the medical apparatus comprises a plurality of wall part sections or consists thereof, which are arranged to be movable relative to each other.

In certain exemplary embodiments according to the present invention, wall part sections are releasably connected to each other.

In some exemplary embodiments according to the present invention, the wall part sections are designed as, or comprise, cylindrical sections which are concentric to each other.

In specific exemplary embodiments according to the present invention, the wall part sections are designed as, or comprise polyhedral sections. In contrast to round cylinder sections, polyhedral sections comprise planes which—in cross section perpendicular to the longitudinal axis—are, define or comprise a polygon. For example, the cross section may be a square or a rectangle with four corners, likewise a polygon, in particular a regular polygon with equal sides on the circumference, with for example, six, eight, twelve, sixteen or more corners. By using polyhedral wall part sections, a relative torsion perpendicular to the longitudinal axis, of the multi-part wall sections towards each other may be prevented in particular when said multi-part wall sections are telescoped completely or partially along a longitudinal direction of the medical device. Moreover, bodies with a polygon cross section are dimensionally more stable than, for example, bodies with a round cross section.

In specific exemplary embodiments according to the present invention, the pressure vessel comprises a first end side and a second end side.

In certain exemplary embodiments according to the present invention, the tubing section and/or the membrane may be adjustable in length and may thus be used for apparatuses of different heights.

In some exemplary embodiments according to the present invention, the medical apparatus comprises at least one outlet which is, or enables, a fluid connection between the pressure chamber and the exterior of the pressure vessel. Further, it comprises a valve, stopcock or other closure device for reversibly closing the outlet or the fluid connection.

The outlet may advantageously be used to lower the pressure present in the pressure chamber by discharging fluid from the latter. This may be necessary or helpful for inserting the limb stump or for adjusting an insertion depth of the stump.

In certain exemplary embodiments according to the present invention, the apparatus according to the present invention is suitable also for treating patients who require a thigh prosthesis. The apparatus according to the present invention may comprise a seating device for treating such patients. This may allow the patient to sit down or to support himself by bony pelvic structures at least in the horizontal direction.

The support device may be releasably or permanently connected to a section of the apparatus or the fluid reservoir, e.g. to its wall, e.g., by plugging on, clamping, screwing or the like.

The support device may have the form of a saddle or a section thereof.

The support device may correspond to the support device, or to a, in particular front, section thereof, as it was filed on 16 Feb. 2015 with the GPTO by the present applicant DE 10 2015 102 185.4 entitled “Aufsitzvorrichtung and Sitzmöbel zum Erstellen eines Gipsabdrucks am sitzenden Patienten zum Fertigen eines Prothesenschafts für die untere Extremität” (Support device and seating furniture for creating a plaster impression on a sitting patient for manufacturing a prosthesis for the lower extremity). The corresponding disclosure of this application is hereby incorporated in its entirety by reference.

In a certain exemplary embodiment according to the present invention, the support device extends in a longitudinal and in a transverse direction thereof. It comprises at least one seat section which extends in the longitudinal direction. The patient may bestride said seat section, i.e. sit as if on a saddle, so that the seat section extends between the thighs from front to rear. It further optionally comprises at least one stopper or stop element (in the following: stopper) extending transversely which is suited to limit the slipping of the patient bestriding the seat section along the support device or along the seat section. This optional limitation is such that the patient may possibly freely slide along the seat section namely in one direction (dorsally, relative to the patient sitting as intended) along the longitudinal direction or the longitudinal axis of the seat section. In the opposite direction (ventrally, relative to the patient sitting as intended), said patient may, however, slide only against the stopper. The stopper is optional. Thus, it does not have to be provided by the present invention. For example, the hand of the orthopedic technician may perform as or replace the function of the stopper. The stopper is preferably comprised by the support device. The longitudinal direction may be the direction which, when the patient has taken place or has been placed on the support device as intended, extends, relative to the patient, from ventral to dorsal or from the front to the back or through the thighs. The transverse direction may be the direction which, when the patient has taken place or has been placed on the support direction, extends, relative to the patient, from caudal to cranial or from the pelvis to the head.

In some exemplary embodiments according to the present invention, the pressure chamber is formed or limited by at least one portion of the wall and by the membrane. The membrane is connected to the section of the wall, preferably in the area of the second end side of the pressure vessel, in a material connection, force-fit connection and/or form-fit connection. There may be a connector, or a connecting device, provided for this purpose. By the connector or the connecting device, an undesirable bulging, moving, floating or extending of the membrane towards the top or into the exterior of the pressure vessel, in which the membrane—unlike in the interior of the pressure vessel—is not laterally supported by the wall, may in some embodiments according to the present invention be prevented or limited to an acceptable dimension. The connector thus holds the membrane, at least substantially, optionally inside the pressure vessel. The latter, or the connector, may counteract an undesirable floating of the limb stump. The floating may have an unfavorable influence on the pressure prevailing in the pressure chamber and by the membrane to the limb stump, in that the membrane no longer contacts the stump with uniform pressure in all sections in which it surrounds the stump. Therefore however, when generating a data model, there are not the optimal pressures put on the stump which results in that the data model has not been generated under later loads, which occur when walking with the prosthesis to be produced. Furthermore, reduced or prevented floating may contribute to protecting the membrane which is in the interior of the pressure vessel protected against damage by the wall of the latter. There is advantageously no need for any other limitation of the floating of the membrane when providing the connector by the present invention, e.g. by a ring which engages tightly on the thigh. Since such a ring would have to be provided in a plurality of sizes in order to be able to generate data models for a plurality of differently thick limb stumps, the skilled person is offered a simplicity that is easy to understand. In addition to simplifying the use of the device, it also means saving in material for rings, costs and the like.

In several exemplary embodiments according to the present invention, a connecting device, optionally annular, surrounding the apparatus holds the membrane by jamming or pressing at the wall, e.g. from bottom towards the top against an optional collar, alternatively or additionally in a radial direction or in a direction perpendicular to the longitudinal axis of the medical apparatus against the wall. The connecting device does not need to have a closed circumference, but may have one.

In several exemplary embodiments according to the present invention, the connecting device is held at the apparatus by a releasable connection, in particular by screws, pins, clamps and/or by clips, a snap-in device or clamping device or the like, which preferably extends horizontally or radially, i.e. preferably perpendicular to a longitudinal axis of the medical apparatus. Alternatively or additionally, other releasable connections are encompassed by the present invention, in particular those which extend parallel to a longitudinal axis of the medical apparatus.

The connecting device may alternatively or additionally be provided in order to clamp the membrane, in a radial direction, against the exterior wall of the wall, optionally even without a collar being provided at the wall. For this purpose, the connecting device may comprise devices for clamping it to for shortening it in a circumferential direction (i.e. which may be shortened in a circumferential direction). If the free end of the membrane now lies between the outside of the wall and the inside of the connecting device, and if there is tension generated by the devices of the connecting device, by which the latter is stretched and/or shortened in circumferential direction, then the connecting device presses the membrane from the outside against the wall (or against another section of the apparatus; the term “wall” is herein exemplarily used). The connecting device thus holds the membrane or the end of the membrane in place by pressure and seals thereby or therewith the membrane against the wall.

In some exemplary embodiments according to the present invention, the connector is connected to the membrane in a section of the membrane which does not lie in the area of the insertion opening.

In certain exemplary embodiments according to the present invention, the membrane is connected to the pressure vessel in the area of the insertion opening or of the first end side of the pressure vessel, and additionally in a second area or section of the pressure vessel which is different therefrom. The second area may preferably be in the interior of the pressure vessel and/or in the pressure chamber. The membrane may preferably be connected to the second area by the connector. The membrane may preferably be in contact only indirectly with the second area, namely optionally by the connector, i.e. preferably not having, itself, a direct contact with the second area, preferably not touching the latter.

In some exemplary embodiments according to the present invention, the second area is a central section or the middle of the bottom surface or of the second end surface or end side.

In some exemplary embodiments according to the present invention, the connector is an elastic spring or comprises an elastic element.

In some exemplary embodiments according to the present invention, the connector is not elastic nor stretchable.

In some exemplary embodiments according to the present invention, the connector is length-adjustable. Said length may be adjusted by a corresponding adjusting device, which may preferably be adjusted from outside the pressure vessel. As a result, the distance between the distal end of the membrane and, for example, the bottom surface, the lower end side or end surface of the pressure vessel is changed. This allows to adjustably arrange the membrane within the pressure vessel which in turn may allow an optimal adjustment of the apparatus according to the present invention with regard to the specific limb stump regardless of its length.

In some exemplary embodiments according to the present invention, the connector directly or indirectly connects the membrane to an end side of the pressure vessel which lies opposite to the end of the pressure vessel comprising the insertion opening, i.e. connecting it in particular to a lower end side, end surface or bottom surface.

In some exemplary embodiments according to the present invention, the connector connects the membrane to a middle or central area of the end surface, end side or bottom surface. This allows or promotes a comparatively straight arrangement of the limb stump inserted into the membrane within the pressure vessel. This arrangement may prevent wrinkles from building up in the membrane and may promote a uniform pressurization. Preventing wrinkles may further be advantageous, since wrinkles may impede a—in particular digital and/or automatic—measurement of the dimension of the limb stump inserted to the pressure vessel or vessel.

In some exemplary embodiments according to the present invention, connecting is to be understood as a form-fit connection and/or force-fit connection and/or material connection.

In some exemplary embodiments according to the present invention, the connector is arranged to connect a lowest section of the membrane, during use, or a central section of the membrane.

In some exemplary embodiments according to the present invention, the connector is arranged to keep a distance within predetermined limits between the section of the membrane connected to the connector on one side and the section of the bottom surface or lower end side or end surface, which is likewise connected to the connector, on the other side. The distance may be, e.g., constant using a non-elastic connector.

In some exemplary embodiments according to the present invention, the membrane is connected directly to a central area of the bottom surface, bottom side or lower end side. The connector may hereby be the result of a joining process, e.g. an adhesive section, a rivet or the like.

In some exemplary embodiments according to the present invention, the membrane touches, in the area of its connection, the bottom surface, bottom side or lower end side; in other embodiments, the membrane or material thereof does not touch the aforementioned.

In some exemplary embodiments according to the present invention, the medical apparatus comprises a support on which the standing patient may support himself, when the limb stump is inserted in the interior through the insertion opening.

In some exemplary embodiments according to the present invention, the medical apparatus comprises a hanging device. It allows to hang up the device for example on the wall, for example for making a plaster impression for a lower-arm prosthesis. The patient can press the arm against the membrane in a specified way.

In some exemplary embodiments according to the present invention, the wall of the pressure vessel limits the pressure chamber. Furthermore, it comprises an inlet which serves for introducing fluid in order to increase the pressure prevailing in the pressure chamber of the pressure vessel. The inlet may be the a.m. outlet or may be a separate device.

In some exemplary embodiments according to the present invention, the membrane is partially, in at least one section thereof or completely or as a whole less than 2 mm thick, preferably less than 1 mm thick.

In certain exemplary embodiments according to the present invention, the pressure vessel comprises a closable air release opening with corresponding closure device. Air can escape via it, in its non-closed state, out of the pressure chamber. This is advantageous when filling the pressure chamber with a liquid for the first time; the apparatus, if it is intended to be filled with liquid, does not have to be purchased or delivered filled with liquid. It can be air-filled and therefore remarkably lighter for transportation. The air present may be released out of the closable air release opening when filling with liquid.

In certain exemplary embodiments according to the present invention, the pressure vessel is designed in one-piece or integrally. This may advantageously effect a reliable tightness of the pressure vessel.

In some exemplary embodiments according to the present invention, the medical apparatus comprises a device by means of which at least the pressure vessel is vertically adjustable relative to an underground on which the apparatus rests or on which the patient is standing with his healthy leg.

In certain exemplary embodiments according to the present invention, the interior and the exterior of the pressure vessel may be respectively separated from each other by a wall.

In certain exemplary embodiments according to the present invention, the apparatus does not comprise an edge or ring with an opening for the patient's thigh, which edge or ring is designed to be releasably connected to the pressure vessel and/or the wall.

In certain exemplary embodiments according to the present invention, the pressure vessel and/or the wall comprise no air opening.

In some exemplary embodiments according to the present invention, the pressure vessel comprises in its interior no struts which are spaced apart from the wall, in particular none which are connected to the membrane and/or in particular none which extend in the longitudinal direction of the pressure vessel.

In certain exemplary embodiments according to the present invention, at least one, preferably central or middle, section of the membrane is attached, preferably releasably, to a section of the wall by at least one connector.

In certain exemplary embodiments according to the present invention, the membrane is made of or comprises a material which comprises, in a first direction and/or in a second direction thereof, fibers embedded in a matrix or otherwise connected thereto.

In certain exemplary embodiments according to the present invention, the membrane, which optionally comprises a matrix, or its matrix is made of, or comprises, silicone.

In certain exemplary embodiments according to the present invention, some or all of the fibers lie in a wavy, curvy or zig-zag pattern.

In certain exemplary embodiments according to the present invention, the membrane is non-stretchable or non-elastic in a first and/or second direction thereof.

In certain exemplary embodiments according to the present invention, “non-stretchable” or “non-elastic” means that the modulus of elasticity of the respective component (connector, membrane, fibers, etc.) is at least above 700 N/mm², preferably above 1000 N/mm², especially preferably above 2000 N/mm².

In certain exemplary embodiments according to the present invention, “non-stretchable” or “non-elastic” means that a extensibility of the respective component (connector, membrane, fibers, etc.) is not more than 20%, preferably not more than 10%, preferably not more than 5%, particularly preferably not more than 2% of its length before the component tears or breaks.

In certain exemplary embodiments according to the present invention, the membrane, fibers thereof and/or the connector has a modulus of elasticity as nylon.

In some exemplary embodiments according to the present invention, the introducing is carried out while the patient is standing.

In some exemplary embodiments according to the present invention, the fluid is a non-compressible fluid, in some exemplary embodiments according to the present invention, the fluid is a liquid, e.g. water.

In some exemplary embodiments according to the present invention, the membrane is deformable to the inside or to the outside, preferably elastically extensible. In others, it is elastic in one direction but it is not elastic or comparatively or substantially less elastic in a direction which is particularly perpendicular thereto.

In some exemplary embodiments according to the present invention, the membrane is arranged such that it can be bulged by the fluid that fills the pressure chamber, through the insertion opening into an exterior of the pressure vessel.

In certain exemplary embodiments according to the present invention, the membrane is extensible, however it remains closed, except for one opening, similar to a finger of a rubber glove or an air balloon in the stretched state.

In some exemplary embodiments according to the present invention, the membrane is arranged on the pressure vessel in order to seal it on the end side.

In some exemplary embodiments according to the present invention, the apparatus has no opening of a connecting tube in an annular or cylindrical space between the, preferably elastically stretchable membrane and the wall of the pressure vessel.

In some exemplary embodiments according to the present invention, the apparatus comprises at least one, preferably two or more cameras, image recording systems, surface scanners, magnetic or laser scanners, 3D scanners, infrared scanners, or other scanners, ultrasound devices, or other devices that are suitable and/or configured to capture or measure the adherent stocking or the sensor arrangement, in which the limb stump is inserted and/or to determine the volume and/or the geometry of the limb stump (e.g. length, width, surface, outer contour, radii, curvatures, dimples, edges, angles etc.). Based on these captures, detections, scanning and the like, it may be possible to manufacture a shaft for the limb stump without having made a plaster impression for it. It does advantageously not require making a plaster impression when using this procedure according to the present invention. In certain exemplary embodiments according to the present invention, capturing or measuring is a scanning of the stump and/or a scanning of the surface thereof.

In some exemplary embodiments according to the present invention, the scanning of the surface of the stump is done with at least one sensor, advantageously with at least one lined structure of sensors, e.g. with sensors arranged in a line or in a row which sensors are arranged for instance along one or several straight lines.

In several exemplary embodiments according to the present invention, the sensors are arranged on a surface, e.g. of the adherent stocking or of the sensor arrangement. Said surface is optionally designed such that it may adapt itself to the surface of the stump for measuring.

In some exemplary embodiments according to the present invention, the surface may consist of or comprise strap-shaped sections. The strap-shaped sections may intersect or overlap, for instance in a midpoint. The midpoint may be equally or substantially equally spaced apart from the free ends of the strap-shaped sections meeting in the midpoint.

The space (alternatively: slot) between two strap-shaped sections which meet or are adjacent at a midpoint, or close thereto, may be free, i.e. in particular free of material. The space (or slot) may increase in width towards the free end or towards the proximal or upper end of the strap-shaped sections during use. In this way, a fold or wrinkle of the sensor arrangement or of the adherent stocking may, in a thin limb stump, be advantageously counteracted or a fold may even be completely avoided.

In several exemplary embodiments according to the present invention, the surface consists of or comprises a preferably even number, such as six, eight or ten, of strap-shaped sections. Each two of them may optionally merge into each other in the area of the midpoint.

In some exemplary embodiments according to the present invention, the width of the strap-shaped sections is 1 cm to 5 cm, preferably exactly or approximately 2 cm.

In several exemplary embodiments according to the present invention, the strap-shaped sections are not elastic.

In some exemplary embodiments according to the present invention, some or all of the strap-shaped sections, in particular those adjacent to each other, are connected to each other by further, preferably elastic, strap-shaped sections or other, preferably elastic, structures.

In several exemplary embodiments according to the present invention, the strap-shaped sections are releasably connected to the further strap-shaped sections or to the other structures.

In some exemplary embodiments according to the present invention, the connection between the strap-shaped sections and the further strap-shaped sections or other structures comprises, or is, a hook-and-loop connection.

In several exemplary embodiments according to the present invention, the strap-shaped sections or the other structures are non-releasably connected to the further strap-shaped sections.

In several exemplary embodiments according to the present invention, the strap-shaped sections or other structures are glued to the further strap-shaped sections.

In several exemplary embodiments according to the present invention, the strap-shaped sections are riveted to the further strap-shaped sections or to the other structures.

In several exemplary embodiments according to the present invention, the strap-shaped sections are at an angle between 70° and 110° with the further strap-shaped sections or with the other structures, preferably at a right angle, for instance measured during the intended use.

In some exemplary embodiments according to the present invention, the strap-shaped sections extend, during the intended use of the sensor arrangement, preferably completely or substantially along the longitudinal axis of the limb stump or in a projection thereon and/or along the outer contour of the limb stump in a direction from distal to proximal.

In several exemplary embodiments according to the present invention, some or all of the strap-shaped sections merge into or are connected with a first connecting section, each of them at a first end (or in a lower area), which distally abuts the limb stump during use.

In some exemplary embodiments according to the present invention, some or all of the strap-shaped sections merge into are connected with a second connecting section, each of them at a second end (or in an upper area), which proximally abuts the limb stump or lies proximal to the first end during use.

The second connecting section may correspond to the retaining device shown in the figures or may be a separate component.

The first connecting section and/or the second connecting section and/or the strap-shaped sections may be made of, or comprise, non-elastic material. This may contribute to ensuring a reproducible position of the sensors towards each other, which sensors are lying on the same or on adjacent strap-shaped sections.

In some exemplary embodiments according to the present invention, signal conductors, power cables or the like extend along the strap-shaped sections. They are connected to the sensors on the respective strap-shaped section in voltage and/or signal communication or may serve such connection during use.

In several exemplary embodiments according to the present invention, the further strap-shaped sections or the other structures extend, during the intended use of the sensor arrangement, preferably completely or substantially transversely to the longitudinal axis of the limb stump and/or along a circumference thereof.

In some exemplary embodiments according to the present invention, the sensors are arranged equidistantly and/or symmetrically on the surface, for example with respect to the optional midpoint.

In several exemplary embodiments according to the present invention, the sensors are arranged in a star-shaped manner, and/or the surface itself is star-shaped.

In some exemplary embodiments according to the present invention, the single sensors are elongated.

In several exemplary embodiments according to the present invention, elongated sensors are arranged according to their length in rows and/or chains.

In some exemplary embodiments according to the present invention, when the limb stump is inserted, the same number of sensors are positioned on several of or on all its cross-sections (preferably in the right angle towards its longitudinal axis), preferably in symmetrical numbers, optionally equidistant to the midpoint of the sensor arrangement. The midpoint may be understood as the point at which the lines of the sensor arrangement converge or as the midpoint of the optional star-shaped arrangement.

In several exemplary embodiments according to the present invention, an opening may be located at the midpoint of the sensor arrangement that is suitable to provide the views to a skin mark therethrough.

The sensor arrangement may be an adherent stocking. It may be a composite of strap-shaped sections. Adherent stocking or composite may each as described herein carry the sensors which are provided for measuring and which are described herein.

In some exemplary embodiments according to the present invention, the optional star-shaped arrangement of the sensors, may enclose the limb stump, when it is inserted, like an octopus, with arms pointing upwards.

In several exemplary embodiments according to the present invention, the preferably star-shaped arrangement of the sensors, e.g. on the adherent stocking, preferably star-shaped or the arrangement which is carrying it is rotated around the longitudinal axis of the limb stump (and, therefore around the midpoint of the sensor arrangement by a few degrees to carry out further measurements in the new position of the sensors, of the arrangement or of the adherent stocking. Thus, with a given number of sensors, which are distributed on the adherent stocking or on the arrangement, a plurality of measuring points or measuring values may be obtained which are more than the number of sensors. This is done with the aim of increasing the accuracy of the fitting of the prosthesis to be manufactured. Further requirements to software and hardware are not made in this case, which keeps the equipment expenditure advantageously low.

In several exemplary embodiments according to the present invention, the adherent stocking according to the present invention is, or comprises, the arrangement of sensors described herein.

In some exemplary embodiments according to the present invention, the arrangement of the sensors comprises a retaining device or is connected thereto. The retaining device is connected for example to the strap-shaped sections, e.g. by gluing, riveting using Velcro connection, by sewing etc., in particular to their ends or free ends, e.g. the ends lying far from the optional midpoint.

The retaining device may be designed to stretch or span the strap-shaped sections or other sections of the arrangement.

In this way, this may contribute having the sensors come to lie at equal distances on the limb stump when it is inserted, e.g. starting from the midpoint.

The retaining device may be a weight which is for example displaceable along the outside of the wall of the pressure vessel of the apparatus, e.g. in form of a ring. The displacement may preferably be done along a longitudinal direction (or parallel thereto) of the apparatus.

The device for measuring, capturing or scanning the limb stump may be configured for obtaining a three-dimensional representation of the limb stump or of a section thereof.

The aforementioned devices for measuring, capturing or scanning the limp stump, such as cameras, imaging recording, scanners, ultrasound devices or other devices, may be provided distributed over the circumference of the pressure vessel or pressure chamber, distributed at identical or different distances on the pressure vessel or around its circumference. They may be integrated into the wall of the pressure vessel. They may be provided to be movable relative to the wall of the pressure vessel, for example in that they can rotate about the pressure vessel or in its interior, along a circumference and/or along a longitudinal direction of the pressure vessel.

In certain exemplary embodiments according to the present invention, the apparatus comprises a rotatable device which can carry and move in a rotating manner the aforesaid devices for measuring, capturing or scanning the limb stump.

The aforesaid devices for measuring, capturing or scanning the limb stump may also include a light source. Thus, for example a light source, which is directed towards the limb stump and illuminates it, and a camera may be rotated together about the limb stump; said light source and said camera preferably being at a fixed distance to each other, (e.g. 10 to 20 cm, e.g. 15 cm).

In some exemplary embodiments according to the present invention, the apparatus comprises at least one device which is configured for generating or calculating a data model of the limb stump or a data model of the shaft to be produced for the measured or scanned limb stump.

The data model, also referred to herein as a model, is preferably three-dimensional. It is preferably continuous.

In some exemplary embodiments according to the present invention, the apparatus comprises a shaping device or is connected thereto in signal communication. The shaping device is arranged and/or configured to manufacture the prosthesis shaft based on the data model of the limb stump or on the data model of the shaft to be produced for the limb stump.

In some exemplary embodiments according to the present invention, the shaping device is a CNC milling device, a rapid prototyping device or a 3D printer (abbreviation for: three-dimensional printer).

The membrane has a top and a bottom. In the use of the apparatus according to the present invention, the top faces the adherent stocking and the limb stump, the bottom limits the pressure chamber. In certain exemplary embodiments according to the present invention, the membrane comprises, at least on its bottom, a marking which is recognizable or identifiable as such through the device for measuring, capturing or scanning the limb stump. The marking may serve as an orientation when calculating the volume or the surface of the limb stump.

This marking may be an optical and/or haptic marking. It may be an elevation, a contrast, a color marking, a coding, a color pattern, a bar code or the like. It may be symmetric or asymmetric. It is preferably applied by the manufacturer on the membrane or integrated therein. It serves to be recognized and/or evaluated by one of the devices of the apparatus. The software used for this purpose may be programmed to identify the marking and/or to evaluate its spatial position.

In several exemplary embodiments according to the present invention, the method according to the present invention for generating a data model of a limb stump encompasses the step of capturing signals of the adherent stocking, wherein transmitting signals to the metallic and/or magnetic particles in the adherent stocking has been carried out prior to the captured signals.

In several exemplary embodiments according to the present invention, the method according to the present invention for creating a data model of limb stump encompasses the step of generating a data model of the limb stump, wherein based on the generated data using mathematical methods, a 3D surface and/or a volume model are generated from the detected signals.

In several exemplary embodiments according to the present invention, the method for creating a data model of a limb stump according to the present invention encompasses the step of transferring the data model to a production facility for manufacturing a prosthesis or a prosthesis shaft, without having to produce a plaster impression as an intermittent step prior to transferring the data.

In some exemplary embodiments according to the present invention, the apparatus comprises no push rod protruding from the pressure vessel, which push rod comprises a coaxial anti-compression cup having an annular wall at its inner wall.

In some exemplary embodiments according to the present invention, the apparatus comprises no carriage or slide on which the pressure vessel would be mounted.

In some exemplary embodiments according to the present invention, the apparatus comprises no device for generating pressure, which device is electric, hydraulic and/or hydraulically operated or operable; in particular no device which is arranged for generating pressure to the membrane or is not connected to the such device.

In several exemplary embodiments according to the present invention, the method according to the present invention encompasses creating a data model based on the data captured using the device for capturing, scanning or measuring the limb stump. Creating a data model may be referred to as generating a data model. The data model may be referred to as model.

The model is preferably a continuous model. It is preferably a three-dimensional model.

In some exemplary embodiments according to the present invention, scanning is done using ultrasound.

In some exemplary embodiments according to the present invention, scanning encompasses a 3D-scanning.

In some exemplary embodiments according to the present invention, the method encompasses manufacturing the prosthesis shaft based on the model. It uses a shaping process for this purpose.

In some exemplary embodiments according to the present invention, the method does not encompass determining and/or using a value of the weight of the patient.

In certain exemplary embodiments according to the present invention, the method does not encompass generating and/or calculating cross-sections through the limb stump or cross-section data.

In some exemplary embodiments according to the present invention, the method does not encompass specifying or considering a target compression.

In some exemplary embodiments according to the present invention, the method does not contemplate covering the distal stump with a cap.

In some exemplary embodiments according to the present invention, the membrane is not tubular, i.e. not open at both ends thereof.

In certain exemplary embodiments according to the present invention, the membrane is made of fluid-tight, in particular water-tight, material. It can be made of or comprise silicone. It can be made of or comprise fiber-reinforced silicone. A co-polymer or a rubber may be provided instead of silicone.

In some exemplary embodiments according to the present invention, the membrane is made of or comprises plastic. A plastic may be, for example polyamide.

In some exemplary embodiments according to the present invention, the membrane is made of rubber or comprises at least one rubber. The rubber may be a synthetic rubber. A synthetic rubber may be, for example, chloroprene rubber. Chloroprene rubber may be referred to as polychloroprene or chlorobutadiene rubber. The synthetic rubber may be foamed.

A rubber membrane may be coated on one or on both sides. The coating may be a textile coating, wherein the textile coating may consist of, or comprise, the plastic polyamide. The coating of the rubber may be referred to as laminating (connecting) the rubber to a textile fabric.

In some exemplary embodiments according to the present invention, the membrane is coated on one or on both sides, completely or partially, for instance with a water-impermeable coating.

In some exemplary embodiments according to the present invention, the adherent stocking may be a liner. A liner may be a textile stocking which in practice is usually pulled over the limb stump.

In several exemplary embodiments according to the present invention, all or some of the sensors are iron cores, preferably with magnetic coils, or comprise magnetic cores, preferably with magnetic coils.

In some exemplary embodiments according to the present invention, the sensors and/or the transmitting device and/or the receiving device are not based on an optical system nor on a laser-based system.

In some exemplary embodiments according to the present invention, the pressure vessel is at least partially made of plastic.

In some exemplary embodiments according to the present invention, the pressure vessel is at least partially made of glass. Glass may be referred to as an amorphous solid. Glass may consist predominantly of silicon dioxide or may comprise it. Glass may also consist of or comprise organic material. A glass of organic material may be a plastic glass, for example acrylic glass.

In certain exemplary embodiments according to the present invention, the transmitting device and/or receiving device for transmitting and/or receiving signals from the sensors of the adherent stocking is not contained and/or integrated in a sensor glove. A sensor glove may be prepared to scan a limb stump in order to measure, for example, anatomical landmarks.

In several exemplary embodiments according to the present invention, some or all of the sensors are disposed on the strap-shaped sections.

In some exemplary embodiments according to the present invention, some or all of the strap-shaped sections, which carry sensors, are connected in one or several, e.g., end-sided sections with at least one other strap-shaped section, likewise carrying one or several sensors, for example, with at least one adjacent strap-shaped section. Such sections are, therefore, also referred to as connection points. However, between such connection points, the connected strap-shaped sections are preferably spaced from adjacent strap-shaped sections.

In several exemplary embodiments according to the present invention, some or all of the strap-shaped sections are connected in a hinged manner or articulated to at least one or several of the other strap-shaped sections, which preferably likewise carry at least one sensor.

The hinged connection of the connection points may, for example by each interconnected strap-shaped section, be designed as pin connection, as sufficiently loosely mounted rivet or in any other way.

The hinged connection in the connection points may be a pivotal connection.

In several exemplary embodiments according to the present invention, some or all of the connecting points, in which connected strap-shaped sections are each connected to each other, comprise at least one sensor.

Embodiments such as those described above, may allow to adjust, in particular to reduce, the distance between sensors to be adjusted in the longitudinal direction of the limb stump. This is an advantageous result due to the scissor-like or diamond-like adjustability of the arrangement of strap-shaped sections, which are connected to each other in a hinged manner by the connection points. In the scissor-like adjustment, rows of sensors, which extend in the longitudinal direction of the limb stump, may approach, or move away from, each other. However, the arrangement of the sensors in single rows remains unchanged. Thus, the adherent stocking or the sensor arrangement may be adjustable in length and thus suitable for measuring differently long limb stumps with one and the same adherent stocking, or sensor arrangement. Patients with comparatively short limb stumps can even benefit from having their limb stump measured with comparatively larger sensor density (in the longitudinal direction of their limb stump).

In some exemplary embodiments according to the present invention, a strap-shaped section is to be understood as a material section, which is longer than it is wide. For example, a strap-shaped section may be an elongated, narrow, strip-like piece whose width is greater than its height.

Some or all of the embodiments according to the present invention may comprise one or several of the advantages mentioned supra or in the following.

It is obvious that the prosthesis shaft is useful to the patient due to the recovered mobility, especially when standing and walking. When standing and walking, when the shaft is loaded according to its intended purpose, the shaft must therefore fit or sit especially comfortably. The methods known so far for producing the plaster impression or for measuring the dimensions of the stump do not adequately comply with or meet or fulfill this, since they do not take into account the soft tissue displacement in the stump, as they occur later when the shaft is subjected to load, e.g. relative to the bony portion of the stump, due to lack of loading the stump during measuring or plastering. The result may lead to inaccuracy in the manufacturing of the shaft, which even by determining the body dimensions at precisely defined heights and ranges lies in the cm-range. This is advantageously not the case when the apparatus according to the present invention is used while the patient is standing—the stump undergoes, during the measuring for the purpose of generating a data model, almost identical loads and soft tissue displacements as in the later load in the shaft.

Using the present invention, it is advantageously possible for the first time with a standing patient loading the stump to reliably and above all reproducibly measure the dimensions of the stump, a data model representing the stump.

Thus, the present invention allows to achieve a stump model as a basis for a shaft that is conveniently fitting the person wearing it, especially when walking and standing. Less skill is needed than hitherto required.

Thus, the present invention enables the manufacturing of shafts for prostheses of human upper and lower extremities in an objective manner based on directly obtained measurement data. The present invention thus enables the manufacturing of a well-adapted prosthesis shaft, wherein, however, the expensive activities, which are executed purely subjectively and manually and which are required in the methods of the state of the art, may be avoided.

Thereby, it is advantageously possible in the present invention and in certain embodiments intended to use a purely external scanning or measuring or imaging. This is considerably less complex, both in terms of the evaluation of their measuring and in the costs for acquiring the required devices.

By the present invention, with a standing patient and thereby having an almost realistically loaded stump during the generating of the data model of a, it is advantageously also possible for the first time to obtain already during the measuring a feedback from the patient concerning pinching or hurting areas or points. Using the apparatus according to the present invention, the condition or situation of the shaft when worn later is so to say already felt “in advance” by the patient during the measurements; unsatisfactorily fitting sections of the later shaft are, therefore, recognized early or anticipated. In this way, desired changes or upholstering may be expressed by the patient already during the measuring and prepared by the orthopedic technician. This may, over the time which passes until the final, fitting shaft is present, (help) saving a significant amount of work and time.

In many exemplary embodiments according to the present invention, there is advantageously no need for an access to a source of electrical voltage, compressed air or a water line. The apparatus may, therefore, be used self-sufficiently and mobile. This applies in some exemplary embodiments according to the present invention to the entire apparatus. In other exemplary embodiments according to the present invention, the pressure vessel comprises no access to a source of electrical voltage, certain devices of the apparatus, e.g. the devices for measuring, scanning etc., are however embodied with an access to a voltage network.

The present invention allows, in contrast to the common methods of the state of the art, to produce shafts for prostheses in an objective manner. This ensures a better supply thanks to an improved fitting form and may reduce the production costs by requiring no or only a small expensive manual processing and adaptation. In addition, the patient care can be accelerated since the time-consuming adaptation steps may, at least in number, be greatly reduced, or even be completely eliminated.

In the following, the present invention is exemplarily explained with regard to the accompanying drawings in which identical reference numerals refer to the same or similar elements. In the partly highly simplified figures, the following applies:

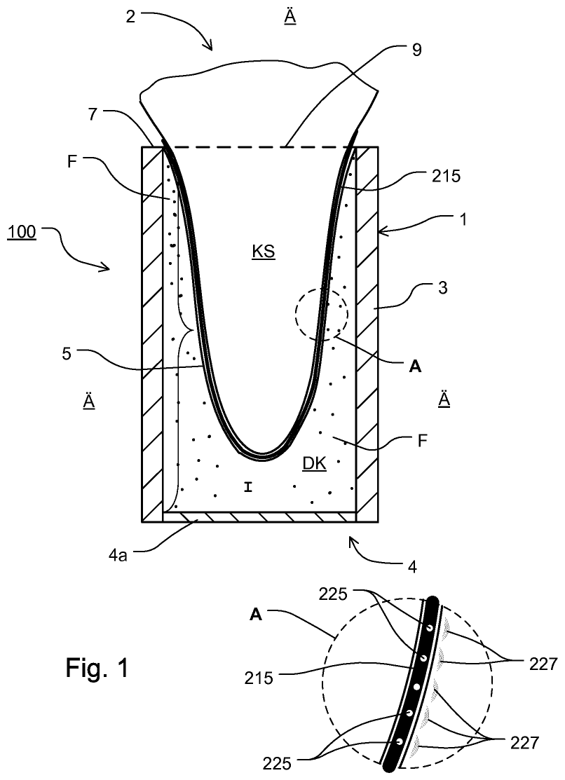

FIG. 1 shows a longitudinally cut medical apparatus according to the present invention;

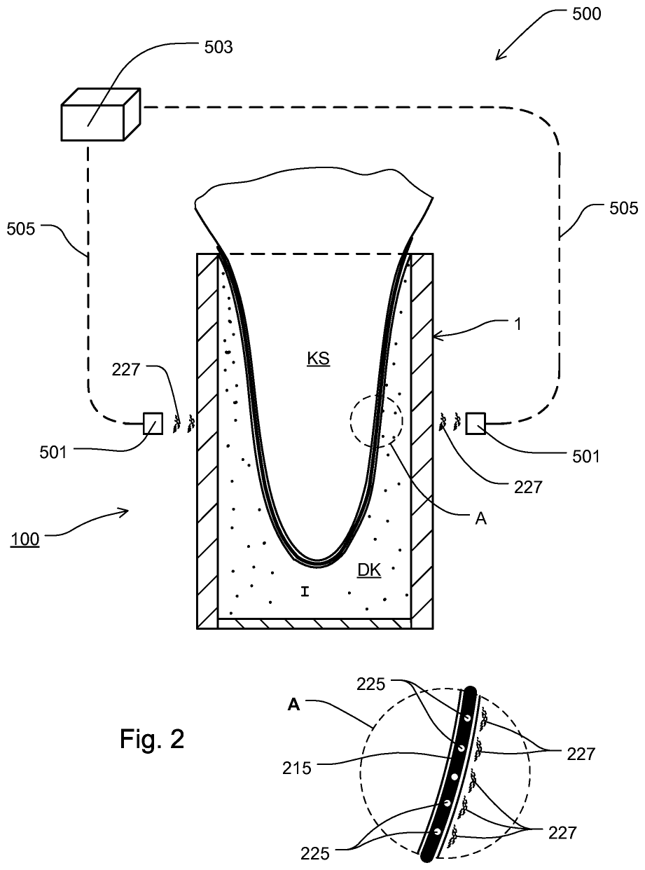

FIG. 2 shows a set according to the present invention with the apparatus according to the present invention, a transmitting device and/or a receiving device and a signal processing device and/or a signal evaluating device;

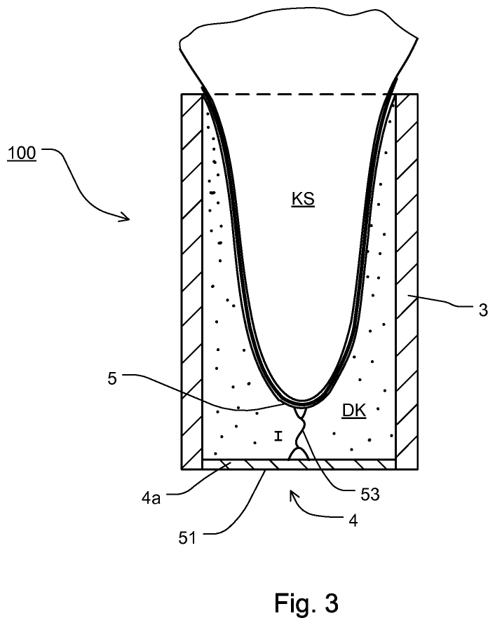

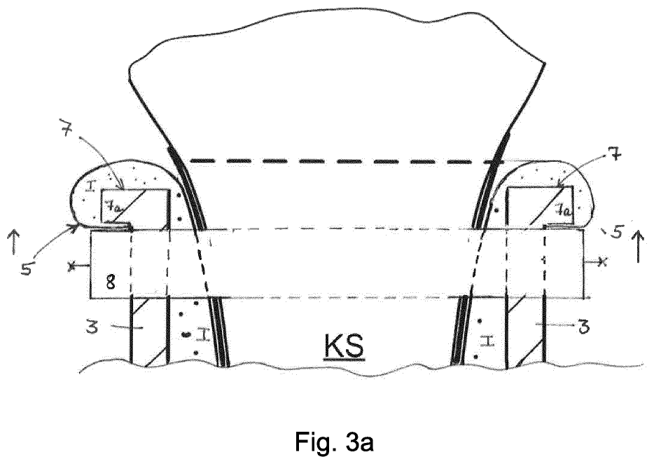

FIG. 3 shows the apparatus according to the present invention with a connector at the lower end area of the membrane;

FIG. 3a shows a way of connecting the membrane and the upper edge of the wall in a possible embodiment of the apparatus according to the present invention;

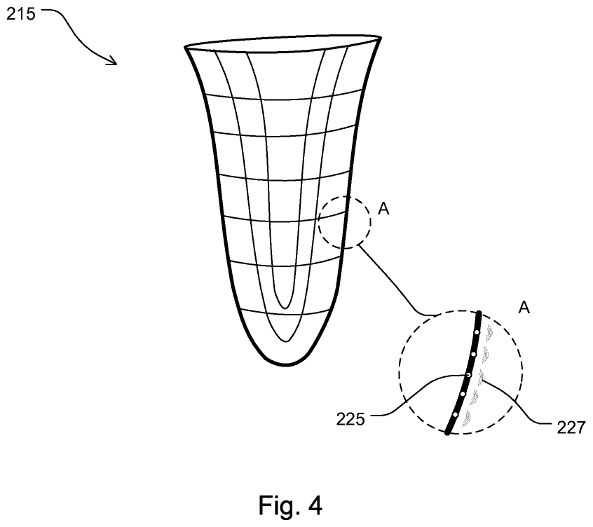

FIG. 4 shows an adherent stocking according to the present invention in a side view;

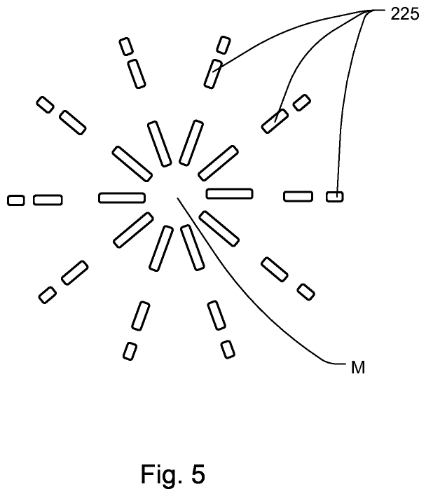

FIG. 5 shows a sensor arrangement according to the present invention on an elastic surface or an elastic carrier;

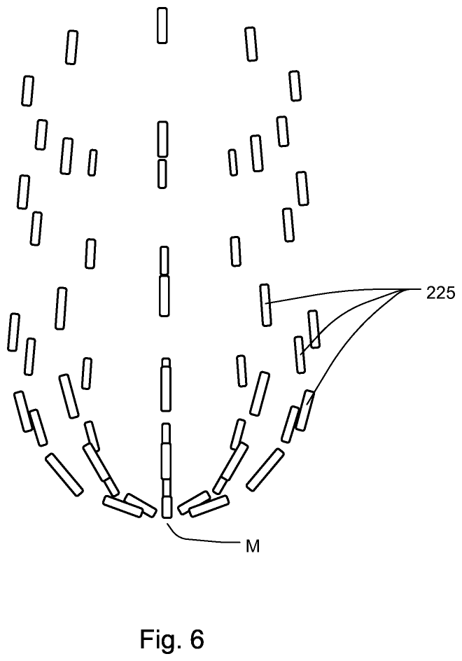

FIG. 6 shows a sensor arrangement as in FIG. 5 with the limb stump being inserted therein, which sensor arrangement contacts the limb stump like an octopus—from below with upwardly facing arms;

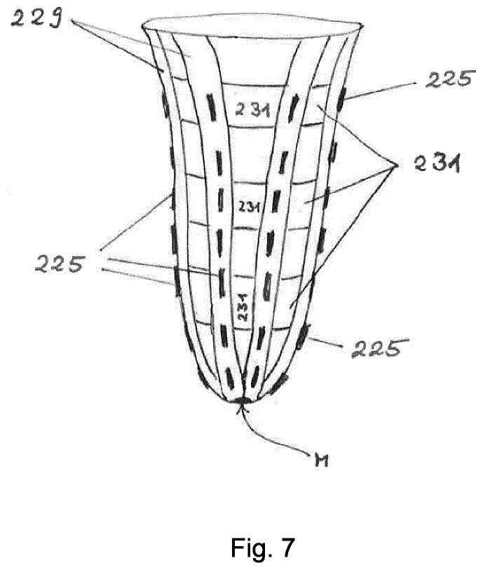

FIG. 7 shows the sensor arrangement of FIG. 5 and FIG. 6 in a further embodiment;

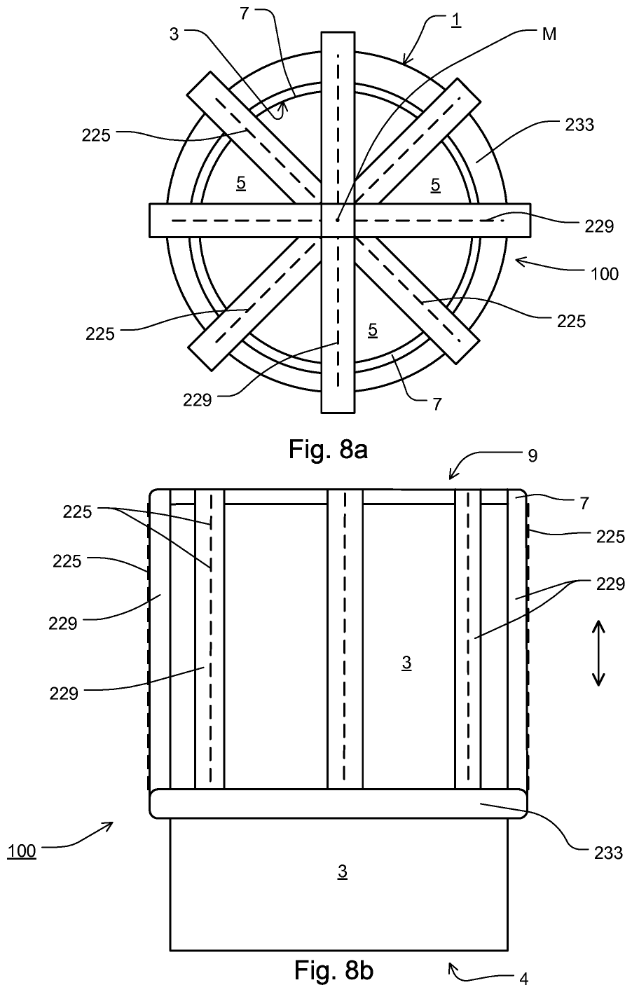

FIG. 8a shows a sensor arrangement according to the present invention in a further embodiment, disposed or applied on a medical apparatus according to the present invention, in a top view on the latter, with a view in its interior;

FIG. 8b shows the medical apparatus of FIG. 8a from the side;

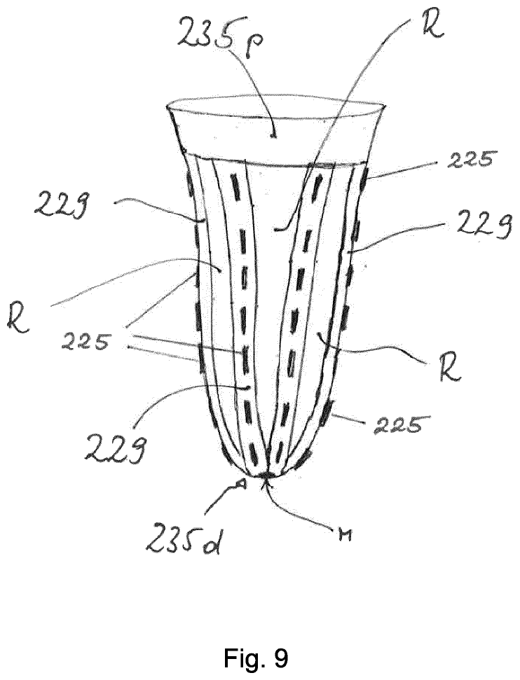

FIG. 9 shows the sensor arrangement according to the present invention in a further embodiment; and

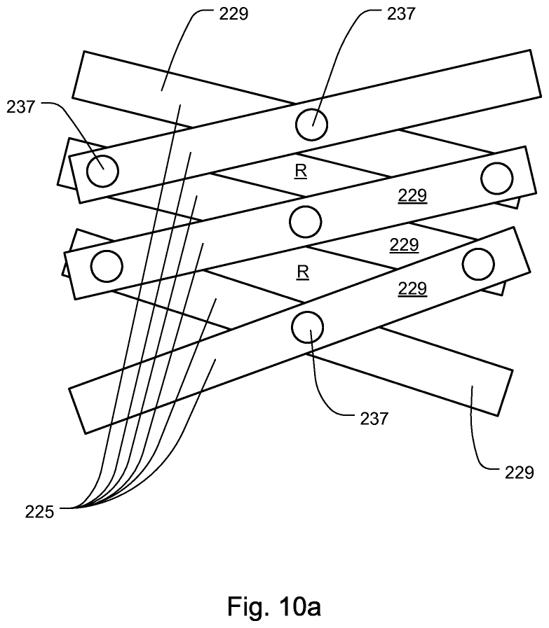

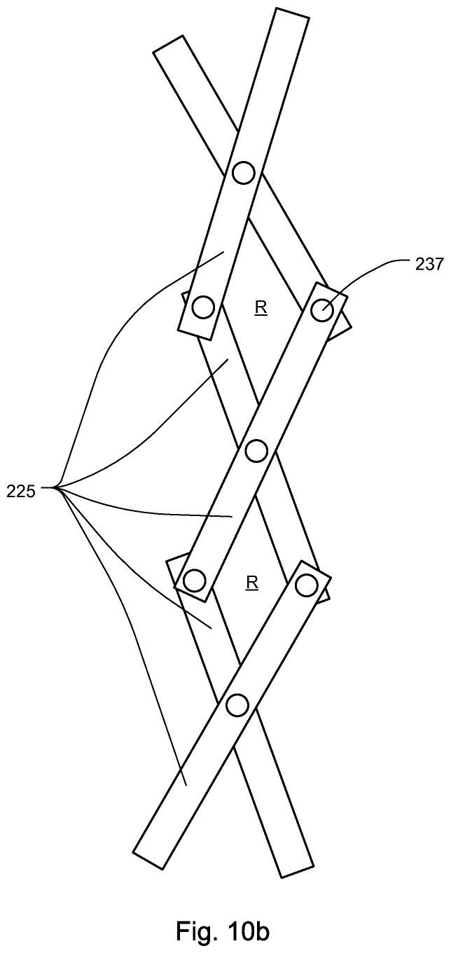

FIG. 10a, b show the sensor arrangement according to the present invention again in a further embodiment.

FIG. 1 shows an exemplary, longitudinally (i.e. with respect to FIG. 1 from top to bottom) cut apparatus 100 from the side. The apparatus 100 comprises at least one pressure vessel 1 with a wall 3, a membrane 5 and an adherent stocking 215.

The pressure vessel 1, shown in FIG. 1 as purely optionally cylindrical, comprises optionally a first end side 2 (at the top in FIG. 1) and a second end side 4 (at the bottom in FIG. 1). The second end side 4 in the exemplary embodiment of FIG. 1 is fluid-tight sealed by a bottom plate or bottom surface 4a against an exterior A. The bottom surface 4a may be made of the same material as the wall 3.

The membrane 5 separates, in a fluid-tight manner, a fluid chamber or pressure chamber DK of the pressure vessel 1 from an exterior of the fluid chamber or pressure chamber DK, or exemplarily from the exterior A, i.e., a surrounding of the pressure vessel 1, or, as shown in FIG. 1, against a limb stump KS inserted into, or surrounded by, the membrane 5.

The membrane 5 may be fluid-tight connected to the pressure vessel 1 at an upper, usually ring-shaped, rectangular, square or differently shaped circumferential edge 7 of the wall 3, or at another site.

The upper edge or rim 7 is situated in a plane in which there is an insertion opening 9 of the pressure vessel 1 or it delimits said insertion opening 9 at its circumference. The insertion opening 9 is situated in the plane which is indicated with a dashed line.

The insertion opening 9 serves for inserting the limb stump KS, which is optionally wrapped with the adherent stocking 215, into an interior I of the pressure vessel 1.

The interior I is the volume delimited by the wall 3 of the pressure vessel 1. It extends from the second end side 4, which is fluid-tightly sealed by the bottom surface 4a, to the insertion opening denoted with 9 and indicated by a dashed line.

The pressure chamber DK is filled with a fluid, here exemplarily with liquid F indicated by points. A filling with gas is also contemplated or covered by the present invention.

In FIG. 1, the apparatus 100 is illustrated in a state in which the extremely schematically-indicated limb stump KS of the standing patient is inserted into the interior I such that it is surrounded by the adherent stocking 215 or by the membrane 5 at least in its distal section. The membrane 5 contacts together with the adherent stocking 215 the limb stump KS like a second skin.

The limb stump KS is preferably weighted or pressed with the full body weight of the standing patient. The amount of the liquid F is measured with regard to the known volume of the interior I or the pressure vessel 1 such that the limb stump KS may enter through the insertion opening 9 into the pressure vessel 1 at least so deep or far that the entire area of the adherent stocking 215 contacts the membrane 5; at least as much as it is relevant for generating a data model of the limb stump KS. At the same time, the amount of liquid F is optionally measured such that the distal end of the limb stump KS (at the bottom in FIG. 1) does not touch the bottom of the pressure vessel 1 or does not support itself on the bottom, respectively. In this way it is ensured that the patient rests with the inserted extremity on the pressure of the fluid and that the adherent stocking 215 undergoes or experiences at each point the same pressure by the membrane 5.

As is shown in the figure, the membrane 5, when no limb stump KS is inserted into the pressure vessel 1, lifts up or floats due to the pressure of the fluid, here of the liquid, and a liquid level is established (not shown in FIG. 1). Hence, the shape of the membrane 5, which is shown in FIG. 1, represents the shape which the membrane 5 takes under pressure when it contacts the inserted limb stump KS and is pulled under elastic stretching by the latter—in the example of FIG. 1—deep into the interior I towards the bottom surface 4a.

It is further to be seen in FIG. 1 that due to the fact that the wall 3 and the membrane 5 both prevent a fluid communication between the pressure chamber DK and the exterior A or prevent a fluid leakage out of the pressure chamber DK, they, hence, allow that the desired pressure builds up within the pressure chamber DK of the pressure vessel 1. However, they do not permit it to escape out of the latter or to be released.

As is seen in FIG. 1, the pressure chamber DK is thus formed by the membrane 5 and at least by parts of the wall 3 which, in this example, include also the bottom surface 4a of the end side 4.

In exemplary embodiments of the present invention unlike those shown in FIG. 1, the pressure chamber DK may consist of or comprise a completely closed membrane, which may lie in the interior I of the pressure vessel 1 like a balloon or a bubble.

The pressure vessel 1, shown in FIG. 1 or in one of the following figures, as well as the pressure vessel of any other embodiment according to the present invention, may comprise a non-round cross section instead of a round one, preferably an edgy cross section. The cross section may exemplarily be rectangular, polygonal or square. Particularly during the automatic measuring of the limb stump KS, when the limb stump is inserted into the apparatus 100, using a camera or the like as described herein, the last-mentioned cross sections may advantageously prevent or diminish artifacts which may be caused by the concave circumferential surface.

The adherent stocking 215 comprises optional sensors 225 which are illustrated in a simplified manner in the detail enlargement A as circular points. The sensors 225 are arranged in the adherent stocking 215 purely exemplarily equidistant from each other, but may likewise not be arranged equidistant and/or be arranged in another arrangement, for example on the inner side and/or on the outer side of the adherent stocking 215. For example, the sensors 225 may be arranged closer to each other in the distal end area (in FIG. 1 below) of the limb stump KS in order to achieve a higher resolution in generating a data model. The sensors 225 may be referred to as detectors, transducers, sensing elements or probes.

The sensors 225 may be active or passive sensors. Active sensors 225 may themselves transmit signals 227 (indicated as wavefront) without having to be activated from the outside. Active sensors may comprise a power supply, for example, they are powered by cables or by power storage elements. Passive sensors 225 may, purely exemplarily, be ferromagnetic iron particles which can be activated (magnetized) by an external magnetic field and subsequently detected.

The sensors 225 may be position sensors or referred to as such. They may transmit signals 227 which solely or in addition to measuring values indicate the position of the transmitting sensors 225.

The sensors 225 may be based on different measuring principles, for example as resistive sensors, inductive sensors, magnetic field sensors, magnetoelastic sensors, capacitive sensors, piezoelectric sensors, optoelectronic sensors, electrochemical sensors or temperature sensors. Alternatively and/or additionally, the sensors 225 may be detected as passive particles by mechanical waves (e.g. ultrasound waves) whose signals 227 may be reflected and, using other sensors outside the pressure vessel 1, e.g. piezoelectric transducers, may be converted into electrical signals and further processed.

By the sensors 225 in the adherent stocking 215, the outer contour of the body stump KS may be determined. Depending on where the sensors 225 are found and where they are arranged in or on the adherent stocking 215, an offset may be determined and included in the further data processing in order to determine the exact outer contour of the limb stump KS. The offset takes into account, for example, the thickness of the adherent stocking 215 and the exact position of the sensors 225 in or on the adherent stocking 215.

The signals 227 emitted by the sensors 225 may be received by the receiving devices (see FIG. 2) outside the apparatus 100. Likewise, passive sensors of transmitting devices (see FIG. 2) may be activated, e.g. by magnetic fields.

FIG. 2 shows an exemplary set 500 according to the present invention with the apparatus 100 according to the present invention, a transmitting and/or receiving device 501, as well as a signal processing and/or evaluating device 503 from the side.

The transmitting and/or receiving device 501 may be designed as a transmitting device 501, as a receiving device 501 or combined as transmitting and receiving device 501. For example, a plurality of transmitting devices 501 for emitting a magnetic field may be arranged on the circumference of the pressure vessel 1. Purely exemplarily, the transmitting device 501, being arranged on the left in FIG. 2, transmits signals 227 in the direction of the adherent stocking 215.

A plurality of receiving devices 501 may be arranged between the transmitting devices 501, above, below or at another point with respect to the transmitting devices 501 in order to receive signals 227 emitted by the sensors 225, see FIG. 2 on the right. These received signals 227 emitted by the sensors 225 may be transmitted, for example via wire or wireless, to a signal processing device 503 and/or to an evaluating device 503 in order to be processed and/or evaluated. The signal path 505 (e.g. by wire or wireless) is indicated by dashed lines.

In the signal processing device 503 or the evaluating device 503, the signals 227 which have been captured by the individual sensors 225 may be further processed and evaluated. For example, the singular signals 227, which may be regarded as the so-called vector-based point cloud, may be further processed to form a surface, in particular a 3D surface. For this, known methods (e.g. rendering) are available. A prosthesis shaft may subsequently be produced using these digital data (vector-based point cloud and/or 3D surface), for example using a 3D printer or a milling machine as a production facility.

FIG. 3 shows the apparatus 100 according to the present invention with a connector 53 at the lower end area of the membrane 5 in an exemplary embodiment.

The membrane 5 is—preferably in its distal, middle or central section or area—connected to a section 51 of the wall 3 or of the end side 4 in a force-fit manner. In the embodiment shown in FIG. 3, the force-fit connection is effected by the connector 53, which extends from a distal end of the membrane 5 to the section 51, here purely optionally, the bottom surface 4a of the pressure chamber DK.

The connector 53 may be a thread, as shown in FIG. 3 by way of example. Any other suitable connector, such as a tape or Velcro or the like, is also encompassed by the present invention.

The connector 53 keeps the membrane 5 connected to the pressure vessel 1 or to the bottom surface 4a, preferably in the area of the second end side 4 of the pressure vessel 1, in particular in the area of the bottom surface 4a and preferably in the midpoint thereof, in a force-fit and/or form-fit and/or material manner.

It is preferred that the connector 53 at least in the state of use of the apparatus 100, that is, when the limb stump KS is inserted into the pressure chamber DK allows a complete or substantially complete flow around the distal end of the limb stump KS. A flow around the limb stump KS is thus still advantageously possible in FIG. 3, except for the surface which corresponds to the cross section of the connector 53. In this way, a floating or a pressure lift also of the distal section of the limb stump KS caused by the fluid may occur unchanged being very important for imitating or sensing the later load conditions in the finished shaft.

In addition to the connector 53 shown in FIG. 3, several or further connectors may be provided. These, like the connector 53, may be connected to the bottom surface 4a. They may alternatively or additionally be connected to the pressure chamber DK or to the pressure vessel 1 at another section of the wall 3 than the end side or bottom surface 4a. This also applies to the connector 53 shown in FIG. 3.

The connection between connector 53 on the one hand and wall 3 or bottom surface 4a on the other hand may be—as the connection, independent thereof, between connector 53 on the one hand and membrane 5 on the other—an adhesive connection, a screw connection, a plug connection, a snap connection a latch connection or the like. It may be releasable or non-releasable.

The connector shown in FIG. 3 is preferably not elastically stretchable. It is preferably not stretchable.

FIG. 3a shows exemplarily a connection of the membrane 5 to the upper edge 7 of the wall 3 of an apparatus 100 according to the present invention. Hereby, the membrane 5 is jammed or pressed by a connecting device 8, which may extend annularly around the apparatus 100, from below against the upper edge 7 of the wall 3. For this purpose, the upper edge 7 may comprise a collar 7a, which e.g. protrudes outwardly beyond the remaining or actual wall 3 of the apparatus 100, as shown in each of FIG. 1 to FIG. 3.

The connecting device 8 may hereby be releasably, in particular adjustably, fixed to the apparatus 100, for example by the screws indicated with “x” in the figure. These may for example extend horizontally and fix the connecting device 8 so firmly to the apparatus 100 such that enough force is generated upwardly in order to tightly connect the membrane 5 with the wall 3 of the apparatus 100, for instance by pressing the membrane 5 from below against the bottom side of the upper edge 7 of the wall 3.

Advantageously, there are no hard edges/surfaces and/or upwardly protruding screws or other fastening devices along the upper edge 7 of the apparatus caused by said connection of the membrane 5 to the wall 3, thus causing, particularly in the crotch area, pressure points and/or pain or may hurt or cause injury.

The membrane 5 lies from above on the upper edge 7 of the wall 3. Thus, said membrane 5 acts, in particular when the pressure is increased in the interior I due to inserting the limb stump into the apparatus 100 by which said membrane 5 bulges outwards and above all over or above the upper edge 7, like a cushion on which the patient my rest with parts of his bony pelvis, or at least with which he may come in contact. Therewith, the comfort for user and/or patient may be increased.

FIG. 4 shows exemplarily an adherent stocking 215 according to the present invention in a perspective side view. In the detail view A, corresponding to the description of FIG. 1, the sensors 225, shown simplified as dots and the exemplarily transmitted signals 227 are visible.

The adherent stocking 215 comprises, in an optional embodiment, at least in sections, an elastic material.

Unlike its representation in FIG. 4, the adherent stocking 215 may be an arrangement of sensors 225 as shown in the following figures.

FIG. 5 exemplarily shows a symmetrical, here also to be donated as star-shaped, arrangement of—here exemplarily elongated—sensors 225 with a view from below.

FIG. 6 shows the sensor arrangement of FIG. 5, attached to a limb stump with a view from the side. The “rays” of the star-shaped arrangement of the sensors 225 (of FIG. 5) lie, during use, onto the limb stump like octopus arms, pointing from bottom to top (with reference to FIG. 6).

In this, it is obvious that preferably the same number of sensors 225 is arranged on one or several cross sections of the limb stump, of the adherent stocking or of the sensor arrangement, preferably in a symmetrical, optionally equidistant, arrangement towards the midpoint of the arrangement M. The midpoint of the arrangement M is herein the point towards which the sensors 225 are aligned. The aforementioned cross sections extend in FIG. 6 or also in FIG. 7 (except a slight perspective view in FIG. 7) substantially horizontally, that is to say from left to right or vice versa with respect to these figures.

An optional arrangement according to the present invention of the sensors 225 on strap-shaped sections which each may comprise a row or chain of sensors 225 and, moreover, may also correspond to the “rays” of the star-shaped arrangement of the sensors, is indicated in FIG. 6.

A data model of the inserted limb stump may be generated by interpolating the sensor information 227 (not shown in FIG. 6, see however FIG. 2) or by a different way.

FIG. 7 shows the sensor arrangement of FIG. 5 and FIG. 6 in a further embodiment. The sensors 225 are hereby arranged on strap-shaped sections 229. As already indicated in FIG. 5, the sensors 225 are arranged on the vertically extending strap-shaped sections 229 as a row or chain, corresponding to the “rays” of the star-shaped arrangement. The “rays” (which extend more or less vertically in FIG. 7) may be connected to further horizontal strap-shaped sections 231 (e.g. two, or three, as shown in FIG. 7, or more). Unlike it is shown in FIG. 7, the horizontal strap-shaped sections 231 may also comprise sensors. The arrangement of these optional sensors on the herein horizontally extending strap-shaped sections 231, may also be in row or chain, however this is not a must.

The further strap-shaped sections 231 may lie over or below the vertically extending strap-shaped sections 229.

FIG. 8a shows a sensor arrangement according to the present invention, placed on a medical apparatus according to the present invention 100 in top view of the latter with view to its interior.

The medical apparatus 100 has optionally a sensor arrangement with sensors 225, which are arranged on the vertically extending strap-shaped sections 229, as in the example of FIG. 7. The sensor arrangement rests on the upper edge 7 of the wall 3 of the medical apparatus 100. In this, the sensor arrangement spans over the insertion opening 9. The view in FIG. 8a is to the interior of the pressure vessel 1 and thus through the strap-shaped sections 229 on the membrane 5. Fastening devices, by which the membrane 5 is fastened to the wall 3, are not shown for reasons of clarity.

It can be seen in FIG. 8a that a retaining device 233 is placed outside to the wall 3, which is connected to a plurality or all of the strap-shaped sections 229 and holds them under tension by retention. The retention may be effected by weight of the retaining device 233. Alternatively or additionally, the retention may be effected by mechanisms such as spring sections, elastic elements or the like.

FIG. 8a thus shows the sensor arrangement on the apparatus 100 before a limb stump KS is inserted into the insertion opening 9.

FIG. 8b shows the medical apparatus 100 of FIG. 8a from the side, likewise before inserting a limb stump KS into the insertion opening 9.

As can be seen in FIG. 8b, sections of the strap-shaped sections 229 lie not only across the insertion opening 229, as shown in FIG. 8a, rather also outside the wall 3, held there in position by the retaining device 233, which here spans the strap-shaped sections 229 by its weight.

If the patient now inserts his limb stump KS into the insertion opening 9 and thus pushes the strap-shaped sections 229 into the depth of the interior of the apparatus 100, then the retaining device 233 moves upwards. In this, sections of the strap-shaped sections 229, which in FIG. 8b still lie outside the wall 3, move therewith into the interior of the pressure vessel 1.

If the patient enters with the limb stump KS into the insertion opening 9 of the pressure vessel 1, then the retaining device 233 lifts. If he pulls his limb stump KS out again, then the retaining device 233 may sink itself again. This is indicated by the double arrow.

The retaining device 233 thus ensures a uniform alignment of the sensors 225 left and right or front and rear on the limb stump KS.

FIG. 9 shows a sensor arrangement being alternative to the sensor arrangement of FIG. 7 in a further embodiment. The sensors 225 are hereby again arranged on strap-shaped sections 229 and optionally, as already shown in FIG. 7, on the strap-shaped sections 229 extending, during use, preferably vertically or from bottom to top along the limb stump KS as row or chain corresponding to the rays of the star-shaped arrangement of the sensors 225, which can be seen, for example, when the strap-shaped sections 229 are spread in a plane starting from the midpoint.

Unlike in FIG. 7, the strap-shaped sections 229 (which extend more or less vertically) are not connected to further, in particular horizontally extending, strap-shaped sections 231 (e.g. two, or three, as shown in FIG. 7, or more), which strap-shaped sections 231 are in FIG. 7 also optional.

However, the strap-shaped sections 229 merge into a first connecting section 235d or are connected therewith, each at a first end of them (in FIG. 9, below), which abuts the limb stump during use distally.

In addition, the strap-shaped sections 229 merge in a second connecting section 235p or are connected therewith each at a second end of them (in FIG. 9 above), which proximally abuts the limb stump or lies proximally to the first end during use.

Between adjacent strap-shaped sections 229 there is respectively a space R. The latter is preferably free, i.e. the front view of the sensor arrangement of FIG. 9 would allow a view through the sensor arrangement also on the strap-shaped sections 229 arranged on the rear-side. However, for the sake of clarity, these rear strap-shaped sections are not illustrated.

The space R increases in width from distal to proximal. It becomes therefore preferably wider to the top (with reference to its use state or to FIG. 9).

In this, adjacent spaces R have the same design. Adjacent spaces R may however also be provided in different sizes or widths.

It is of particular advantage when the design or size of all spaces R is known and stored in a software of the signal processing device.

The first connecting section 235d and the second connecting section 235p jointly define or determine the respective space R. For this purpose, the first connection section 235d and the second connecting section 235p are not elastic. Thus, the predetermined distance between the adjacent strap-shaped sections 229 remains preferably advantageously unchanged, and the positions of sensors 225, which lie on adjacent strap-shaped sections 229 remain likewise unchanged in this respect. The latter may play an essential role in the evaluation, using the signal processing device 503, of the signals emitted by the sensors 225 and received by the receiving device 503.

In FIG. 9, the first connecting section 235d is shown as a material-fit transition of adjacent strap-shaped sections 229. In other embodiments, the first connecting section 235d is itself designed as a separate component, e.g. as a strap structure or cap, which is connected to the adjacent strap-shaped sections 229 in a form-fit and/or force-fit connection.

FIGS. 10a and 10b show the sensor arrangement according to the present invention in again a further embodiment.

In this, the strap-shaped sections 229, which carry sensors 225 are in one or several, e.g. end-sided and/or middle, sections, herein referred to as connecting points 237, connected to at least one other strap-shaped section 229 likewise carrying one or several sensors 225, for instance at least to an adjacent strap-shaped section 229.

In between such connection points 237, the strap-shaped sections 229 connected therewith, are, however, preferably spaced from adjacent strap-shaped sections 229, indicated by the space R in FIGS. 10a and 10b.

In the example of FIGS. 10a and 10b, the connecting points 237 are hinged connections between strap-shaped sections 229. In this, the strap-shaped section 229 may be connected to one, two or more further strap-shaped sections 229.

In the example of FIGS. 10a and 10b, some or all of the strap-shaped sections 229 comprise, each, one or several sensors 225. These may at the same time also represent the connecting points 237 or vice versa. This is not shown in FIGS. 10a and 10b.

The comparison of FIG. 10a to FIG. 10b shows the scissor-like or diamond-like adjustability of the arrangement of strap-shaped sections 229 by the connecting points 237.

The concentration of the sensors 225 in longitudinal direction of the limb stump, which is not shown in FIG. 10a or 10b, but would extend during use of the sensor arrangement in up-down direction, may thereby be larger or smaller.

### LIST OF REFERENCE NUMERALS

100 apparatus
1 pressure vessel
2 first end side
3 wall
4 second end side, may optionally be closed or sealed by the bottom surface or plane
4a bottom surface or plane
5 membrane
7 upper edge
7a collar
8 connecting device
9 insertion opening
53 connector
215 adherent stocking
225 sensors (of the adherent stocking or of the sensor arrangement)
227 signals/sensor information
229 strap-shaped section
231 further strap-shaped section
233 retaining device
235d first, distal connecting section
235p second, proximal connecting section
237 connecting point
500 set with apparatus and transmitting device and/or receiving device
501 transmitting device and/or receiving device
503 signal processing device and/or signal evaluating device
505 signal path
DK pressure chamber of the pressure vessel
I pressure vessel interior
F fluid or liquid
A pressure vessel exterior
KS limb stump
M midpoint of the sensor arrangement
R space (or slot or gap)

1. A medical apparatus for use in generating a data model of a limb stump, in particular of a lower leg stump; wherein the apparatus comprises at least:
a pressure vessel with a fluid chamber or a pressure chamber for receiving or storing a fluid, in particular one being under pressure, wherein the pressure vessel comprises a wall made of a first material, wherein the wall limits an interior of the pressure vessel against an exterior, wherein the pressure vessel comprises an insertion opening for inserting the limb stump into the interior of the pressure vessel; and
a fluid-impermeable membrane made of a second material, which membrane is arranged to form or limit the fluid chamber or the pressure chamber; and
an adherent stocking or another sensor arrangement, to be pulled over the limb stump, wherein the adherent stocking or the sensor arrangement comprises sensors for generating the data model.

2. The medical apparatus according to claim 1, wherein the sensors are at least one of magnetic and metallic particles.

3. The medical apparatus according to claim 1, wherein the sensors are position sensors.

4. The medical apparatus according to claim 1, wherein the pressure vessel is at least partially made of plastic.

5-13. (canceled)

14. The medical apparatus according to claim 1, having an arrangement of sensors, wherein the sensors are suitable to capture data over the surface of a limb stump, and wherein the arrangement of the sensors is star-shaped.

15. The medical apparatus according to claim 1, having an arrangement of sensors, wherein the sensors are suitable to capture data over the surface of a limb stump, and wherein the sensors are elongated and are arranged lengthwise in rows or chains.

16. (canceled)

17. A set having
at least one medical apparatus according to claim 1; and
at least one of a transmitting device for transmitting and a receiving device for receiving signals of the sensors present in the adherent stocking or in the sensor arrangement.

18. The set according to claim 17, having at least one of a signal processing device for processing the signals of the sensors, and an evaluating device for evaluating the signals of the sensors.

19. A method for generating a data model of a limb stump, the method encompassing the steps:
providing a medical apparatus according to claim 1;
detecting signals of the adherent stocking or of the sensor arrangement;
generating a data model of the limb stump.

20. The method according to claim 19, encompassing the further step of transferring the data model to a production facility for producing a prosthesis stump.

21. The method according to claim 20, wherein the production facility is a 3D printer or a milling machine.

22. An adherent stocking or sensor arrangement to cover or to be pulled over a limb stump, wherein the adherent stocking or the sensor arrangement comprises sensors for generating a data model.

23. (canceled)

24. The adherent stocking or sensor arrangement according to claim 22, wherein at least one of the adherent stocking and the arrangement of the sensors on the adherent stocking or on the sensor arrangement are star-shaped.

25. The adherent stocking or sensor arrangement according to claim 22, wherein the sensors are elongated and are arranged lengthwise in rows or chains.

26. (canceled)

27. The adherent stocking or sensor arrangement according to claim 22, wherein at least one of the adherent stocking and the arrangement of the sensors is connected to a retaining device.

28. The adherent stocking or sensor arrangement according to claim 22, wherein the adherent stocking comprises an arrangement of sensors.

29. The adherent stocking or sensor arrangement according to claim 22, having strap-shaped sections, wherein at least some of the sensors are present on the strap-shaped sections.

30. The adherent stocking or sensor arrangement according to claim 29, wherein at least some of the strap-shaped sections, which carry sensors, are connected to each other preferably at their ends, however in other sections they are spaced apart from the adjacent strap-shaped sections.

31. The adherent stocking or sensor arrangement according to claim 29, wherein at least some of the strap-shaped sections are connected to at least one further strap-shaped section in a hinged manner.

32. The method according to claim 19, further comprising:
rotating the adherent stocking or the sensor arrangement about its longitudinal axis, for instance about a midpoint, and again detecting signals from sensors of the adherent stocking or of the sensor arrangement.
